(19) United States
(12) Patent Application Publication
(10) Pub. No.: US 2009/0130256 A1
(43) Pub. Date: May 21, 2009

(54) DECOMPOSITION OF FERMENTATION-INHIBITING SUBSTANCES FROM A FLUID

(75) Inventor: Christian Uphoff, Aschau im Chiemg. (DE)

Correspondence Address: BOYLE FREDRICKSON S.C., 840 North Plankinton Avenue, MILWAUKEE, WI, 53203, US

(73) Assignee: Georg Fritzmeier GmbH & Co. KG, Grosshelfendorf (DE)

(21) Appl. No.: 12/282,284

(22) PCT Filed: Mar. 9, 2007
(86) PCT No.: PCT/DE2007/000434

§ 371 Date: Dec. 2, 2008

(30) Foreign Application Priority Data
Mar. 9, 2006 (DE) 10 2006 010 985.6

(51) Int. Cl. A23C 21/02; C12P 1/00; C12P 7/06; C12G 1/022; C12M 1/00

(52) U.S. Cl. 426/15; 435/274; 435/161; 435/294.1; 426/41

(57) ABSTRACT

There is disclosed a method of decomposing fermentation-inhibiting substances in a fluid including saccharide-containing substances, wherein a microbiotic mixture, especially a mixture of photosynthetically working micro-organisms and light-emitting micro-organisms is introduced into the fluid, the mixture being intended to decompose the fermentation-inhibiting substances.

### BACKGROUND OF THE INVENTION

1. Field of the Invention

The present invention relates to a method of decomposing fermentation-inhibiting substances from a fluid, a method of preparing ethanol, especially from whey, as well as an arrangement of bioreactors for carrying out the method of preparing ethanol from whey.

2. Description of the Related Art

It is known from prior art that ethanol can be obtained from saccharide-containing substances, especially sugar and/or glucose. Cereals, sugar beet, potatoes and whey are known as suppliers of glucose which are suited for the preparation of ethanol. But also other saccharide-containing food and mainly food residues can be fermented.

It is a problem, however, that, on the one hand, by reason of starting disintegration caused by mould or the like, for instance, exactly in food residues fermentation-inhibiting substances are found which impede the preparation of ethanol from these products and therefore make it unprofitable. On the other hand, exactly the preparation of ethanol from whey has not been profitable so far, because the yield of ethanol obtained from whey is not sufficient for a commercial exploitation.

Therefore, it is the object of the present invention to provide a method by which fermentation-inhibiting substances can be easily removed and which thus ensures an as simple and inexpensive preparation of ethanol as possible, especially from whey.

### SUMMARY OF INVENTION

A method of decomposing fermentation-inhibiting substances from fluids including saccharide-containing substances, characterized in that a microbiotic mixture of photosynthetically working micro-organisms and light-emitting micro-organisms is introduced into the fluid, the mixture decomposing the fermentation-inhibiting substances, a method of preparing ethanol, a method of preparing ethanol from whey as well as an arrangement of bioreactors for preparing ethanol from whey.

The present invention is based on the finding that the low yield of ethanol during the preparation of ethanol from saccharide-containing food and/or food residues, such as whey, has to be traced back to two problems:

On the one hand, when using whey as a food residue, that is to say as waste product from cheese factories, inhibitors which on the whole strongly restrict the preparation of ethanol are provided in the whey. These inhibitors include especially copper which renders fermentation of whey into ethanol almost impossible, but also moulds which settle when the whey is not immediately further processed. Such inhibitors are also provided in other saccharide-containing fermentable food, such as grapes or barley, and for the preparation of ethanol have to be removed from the fluid to be fermented.

To this end, a microbiotic mixture of photosynthetically working micro-organisms and light-emitting micro-organisms is applied to the fluid to be fermented, which mixture is intended to decompose the fermentation-inhibiting substances. If the whey is not yet strongly loaded, an addition of hop may also have a sufficient bacteriostatic effect.

In a further preferred embodiment the microbiotic mixture is not directly added to the fluid, however, but the substances from which the fluid is prepared are pre-treated with the microbiotic mixture. This is advantageous especially in producing wine or beer, when the grapes or apples or the brewing barley, respectively, are treated with the microbiotic mixture before preparing must or mash. In this way fermentation-inhibiting substances, such as mildew for instance, can be removed already prior to the fermenting process and cannot contaminate the must or mash, either.

Tests have demonstrated that moreover the subsequent fermenting operation can be definitely accelerated by adding the microbiotic mixture to the must, but above all also when pre-treating grapes with the microbiotic mixture, wherein furthermore a rapid decomposition of sugar and a rapid increase in temperature during fermentation has been observed. Moreover, the addition of the microbiotic mixture could prevent a stale or bad smell of the fermenting product.

It is especially advantageous when the photosynthetically working micro-organisms contained in the microbiotic mixture are prochlorophytes, cyano bacteria, green sulphur bacteria, purple bacteria, chloroflexus-similar forms, heliobacteria and heliobacillus-similar forms, as well as mixtures from two or more thereof and the light-emitting micro-organisms contained in the microbiotic mixture are photobacterium phosphoreum, vibrio fischeri, vibrio harveyi, pseudomonas lucifera or beneckea or mixtures from two or more thereof. Moreover, it may be of advantage when the microbiotic mixture additionally contains plant extracts, enzymes, nutritional trace elements, polysaccharides, alginic derivatives and/or other micro-organisms either individually or in combination.

The other problem existing especially in the preparation of ethanol from whey resides in the fact that the glucose contained in the whey is not freely available. That is to say, it is not possible to simply add yeast to the whey for a fermenting process, but first the glucose must be obtained from the lactose. But lactose is usually contained in the whey only at a percentage of 30 to 40% and moreover does not consist of 100% glucose but also of galactose. Although galactose also is a monosaccharide, it cannot be fermented into ethanol, whereby the yield of ethanol is further reduced.

According to the invention, therefore a method is suggested in which in a first step such inhibitors such as copper, for instance, are removed from the whey. In a subsequent step the lactose provided in the whey is split into glucose and galactose, wherein the latter is not treated like a waste product but is converted into glucose with the aid of a particular yeast strain, the so-called Kluyveromyces, as is shown in a further especially preferred embodiment.

Only then, in a third step, a fermenting agent, especially yeast or bacteria, is added in order to convert the now comparatively high concentration of glucose into ethanol.

Since, as described in the foregoing, especially copper was identified as inhibitor for preparing ethanol, copper is removed from the whey by means of electrolysis in a particularly preferred embodiment.

It is another restriction that during fermentation into ethanol the percentage of ethanol in the fermenting solution has to be observed, because too high alcohol content has a negative influence on the fermenting process. Therefore, as is shown in another advantageous embodiment, the alcohol content is kept at a particular level, preferably at less than 12%. To this effect, preferably excessive ethanol can be discharged from the fermenting solution by means of membrane filtering.

It is especially advantageous when the method according to the invention takes place in a bioreactor. The three principal steps of the method can be carried out successively in the same bioreactor. However, for this purpose the bioreactor has to be prepared for the next step between the individual steps.

Since this is complicated, the method can also take place, as is shown in a further preferred embodiment; in preferably three bioreactors arranged one behind the other, wherein conversion of the individual bioreactors is superfluous due to the method steps fixedly assigned to them.

A first bioreactor is preferably equipped with an electrolysis means so that removal of copper from the whey is made possible. To this end, a specific equipment of the bioreactor can be used. A particularly preferred bioreactor consists of a coated tank and a coated packing, wherein the coatings are selected such that when an electric field is applied the bioreactor itself acts as electrolysis apparatus. A photocatalytic coating of the bioreactor and an active charcoal coating of the packing have turned out to be especially advantageous. When applying the electric field, the packing then serves as anode and the copper ions settle at the active charcoal layer.

Into this first bioreactor whey from a cheese factory, for instance, is introduced and the disturbing copper is removed therefrom. If the whey is strongly loaded, for instance because it was stored already, also the microbiotic mixture can be added to the whey. If the whey is only little loaded or is fresh, also hop can be added to the whey before introducing it into the first bioreactor, preferably 100 g hop per hectoliter of whey so that, on the one hand, already existing load is reduced or at least decelerated, and it can be ensured, on the other hand, that such load does not occur at all.

The whey prepared in this manner is then transferred into a second bioreactor in which the lactose contained in the whey is split up into glucose and galactose by means of lactase. For this purpose, in the second bioreactor moreover Kluyveromyces yeasts can be provided which, on the one hand, equally cause a splitting of lactose into glucose and galactose, but are also adapted, on the other hand, to transform galactose into glucose. In a third bioreactor into which the glucose mixture is introduced, microorganisms, especially yeasts or fermenting bacteria are provided which cause a conversion of glucose into ethanol by means of alcoholic fermentation. The thus produced ethanol is then preferably separated from the glucose mixture by means of a membrane.

Especially preferred is a bioreactor arrangement in which the individual bioreactors are coated with a photocatalytically active layer and have one or more recesses through which the whey passes into the interior of the bioreactor. Moreover, it is advantageous when a packing including active charcoal for enlarging the reaction surface is provided in the bioreactor, wherein preferably copper separates at the packing or microorganisms settle for ethanol preparation and enzymes can be immobilized. Especially advantageous is an embodiment of the bioreactors in which a photocatalytic coating in the form of longitudinal strips alternates with a diamond coating in the form of longitudinal strips.

These, and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

Hereinafter especially preferred embodiments of the invention are explained in more detail by way of figures. The figures are merely exemplary and are not intended to be used for restricting the scope of the claims to the illustrated embodiments.

They show in:

FIG. 1: a schematic drawing of a preferred embodiment of the present invention in which three bioreactors are connected in series so as to carry out the method of preparing ethanol from whey according to the invention; and

FIG. 2: a schematic drawing of a preferred embodiment of a bioreactor for the method according to the invention.

FIG. 3: a schematic drawing of a further embodiment of the present invention so as to carry out the method of preparing ethanol from whey according to the invention.

### DETAILED DESCRIPTION OF THE DRAWINGS

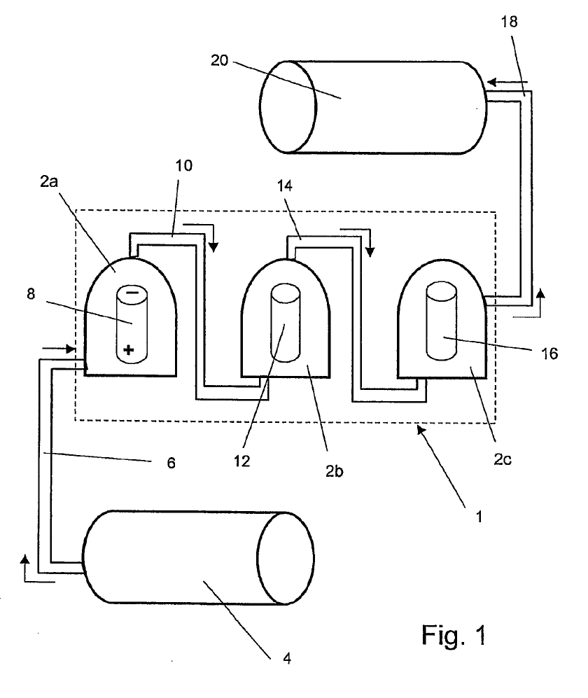

FIG. 1 shows a preferred embodiment for an arrangement 1 of bioreactors 2a-c for implementing the method according to the invention.

In order to prepare ethanol from whey, whey which otherwise occurs as a waste product from cheese factories is further treated. For this purpose, the whey is transferred from a storage or supply tank 4 via a first pipe 6 into the arrangement 1 of bioreactors 2a-c. It is also possible, however, to provide such plant or at least a bioreactor for ethanol preparation directly at the cheese factory. Then the whey could be directly introduced from the cheese factory into the arrangement 1 via the pipe 6 without a tank 4 being required.

In order to prevent bacteriological load of the whey or to reduce the same, in the tank 4 hop, preferably 100 g hop per hl of whey can be added to the whey. Hop has a mycotically selective effect and suppresses the Gram-positive bacteria, whereby foreign germs contained in the whey cannot further multiply. In addition, this has the effect that above all the sugar-metabolic bacteria which reduce the ethanol yield due to the reduction of the sugar content are prevented from multiplying. Hop can be added as total substance, but it is also possible to add only the β-acids of hop necessary for the bacteriostatic effect of the hop as hop extract of the whey.

If the whey is already strongly loaded with foreign germs, it is moreover advantageous to add to the whey a microbiotic mixture of photosynthetically working micro-organisms and light-emitting micro-organisms. This microbiotic mixture is also suited for eliminating moulds existing in the whey.

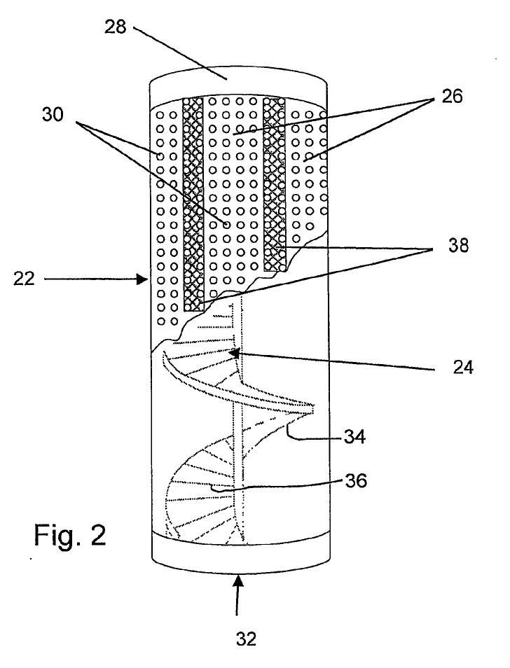

In a first bioreactor 2a the whey is separated from inhibitors for the ethanol preparation. This can take place, especially when copper is present, via electrolysis. In addition, the microbiotic mixture can be added in the bioreactor 2a. For the electrolysis, an electrolytic device 8 is provided in which the copper ions are separated at the anode. The electrolytic device may also be in the form of a coated bioreactor including a packing in which an electric field is formed by an alternate coating of diamond and titanium oxide, for instance, and an active charcoal coating of the packing. The packing may be in the form of a spindle which in turn has a coating of active charcoal. A schematic representation of such a bioreactor including a packing is described in detail in FIG. 2.

Since this electric field is rather weak, however, an external power source should be used for the electrolysis to obtain a good result. For this purpose, a weak current of between 400 and 600 mA or 0.3 to 2.6 V, preferably 1.9 V, is applied and the packing is used as anode.

Since the copper ions are not freely provided in the whey but are bound to the lactase enzymes already contained in the whey and responsible for the splitting of lactose into glucose and galactose, it is reasonable to recover these enzymes so that not an unnecessary plurality of enzymes has to be used in the successive method step of lactose splitting. To this end, the applied current is switched off and a voltage reversal is initialized, whereupon the enzymes detach from the packing, while the copper ions still remain at the packing. If, at the same time, the whey treated in this manner is removed from the first bioreactor 2a via a further pipe 10, the lactase enzymes but no or almost zero copper ions are provided in the whey.

If the whey is directly supplied from the cheese factory, it usually has a temperature of 45° C. to 55° C. For the electrolysis the temperature is insignificant so that the bioreactor 2a need not be equipped with a temperature control unit.

In addition, the whey can be further concentrated in the bioreactor 2a. That is to say, excessive water is removed from the whey so that a high lactose concentration is obtained.

If the whey is processed, it is introduced via the second pipe 10 into a further bioreactor 2b. There lactose is split up into its two components of glucose and galactose. This is preferably done at a temperature of 30° C. to 35° C. Unless the whey has already been cooled to this temperature, because it comes directly from the cheese factory, for example, or it is still too cold, a refrigerating or heating unit (not shown here) which sets the whey to the desired temperature before splitting up the lactose has to be connected ahead of the bioreactor 2b or has to be integrated in the bioreactor. Furthermore, also in the bioreactor itself a temperature sensor (not shown here) can be arranged which constantly controls the reaction temperature.

Enzymes—the lactase as it is called—are in charge of the lactose splitting itself which advantageously are immobilized in a packing contained in a tank 12 accommodated in the bioreactor. The exact structure of the bioreactor is described in FIG. 2. The packing, on the one hand, enlarges the reaction surface and, on the other hand, provides a simple possibility of conversion, even when only one single bioreactor is used, by a simple exchange of the packings from the bioreactor for step B—namely the splitting of lactose—to a bioreactor for step C—ethanol preparation.

Lactase is used as enzyme which is advantageously provided by Kluyveromyces yeasts. The great advantage of using Kluyveromyces yeasts resides in the fact that by the lactase provided by the latter not only a splitting of lactose into glucose and galactose is obtained but also a transformation from galactose into glucose is possible. This is of crucial advantage to the ethanol preparation, because in this way the content of glucose and thus the amount of ethanol is increased.

Advantageously less than 10 mg/l of enzymes, in the optimum case 1 mg/l of enzymes are added to the whey. During an advantageous period of 8 to 10 hours the enzymes then split lactose into glucose and galactose. During a further period of 14 to 16 hours then also galactose is transformed into glucose.

As a result, a relatively highly concentrated glucose mixture is obtained which, in the embodiment illustrated here, for further treatment is transferred into a further bioreactor 2c via a third pipe 14. As mentioned already in the beginning, also the bioreactors 2b and 2c may be integrally formed. In the same the packings would now be exchanged so as to convert the bioreactor to the fermenting process.

The bioreactor 2c is equipped with a packing 16 which may contain yeast or bacteria as micro-organisms used for fermentation. As the two fermenting processes do not take place at the same temperatures, a simultaneous presence of the two types of micro-organisms is possible in the packing, to be sure, but only those micro-organisms for which the appropriate temperature is applied are active, however. In this way an alcoholic fermentation by means of yeasts takes place at a temperature of less than 25° C., preferably between 8° C. and 10° C., while the fermentation with the aid of bacteria, especially thermoanaerobacter ethanolicus, takes place at 60° C. to 65° C. In order to exactly control the temperature a refrigerating or heating unit (not shown here) can be connected ahead of the bioreactor 2c or can be integrated in the same.

Likewise a temperature measuring device may be provided for controlling if the temperature meets the requirements.

The fermenting micro-organisms are immobilized by the packing 14 in the bioreactor 2c which simultaneously makes available an enlarged reaction surface.

Since the fermenting process is inhibited by a too high concentration of alcohol, the already produced ethanol has to be removed from the bioreactor 2c. This can advantageously be done via a diffusion membrane not shown here. But also the use of a specific distilling membrane is imaginable, which is relatively expensive, however. In the ideal case the alcohol concentration should not exceed 12%.

Subsequently, the ethanol prepared can be supplied to a storage tank 20 via a further pipe 18.

FIG. 2 illustrates a schematic representation of a preferred embodiment of the bioreactor consisting of a container 22 and a packing 24. In this embodiment the container 22 has a cylindrical shape, but it may also have any other shape. The side walls of the container 22 are made of stainless steel in the shown embodiment and can be partially provided with a photocatalytically active coating 26. This coating 26 may be formed at the inner circumferential wall of the container 22 and/or—as shown in FIG. 2—at the outer wall 28. In the shown embodiment the container 22 is made of V4A steel and is provided with a titanium dioxide coating. Instead of said titanium dioxide also indium tin oxide or the like may be utilized. The outer wall 28 of the container 22 is provided with a plurality of breakthroughs 30 so that the whey to be converted can pass into the interior of the container 22. Said breakthroughs 30 can be punched, for instance, wherein it is of advantage when the punching burrs then protrude to the inside. The lower end face 32 of the container can be closed so that the whey flows into the container 22 substantially in radial direction. The upper end face can equally be closed. In case that the upper surface lies above the liquid level, closing can be renounced.

In the interior of the container 22 an exchangeable packing 24 is accommodated which, as shown in the front view, has a helical structure. In the shown embodiment said packing 24 consists of a support 34 which may be a helical stainless steel sheet, for instance. A foam material, for instance PU foam, is applied on both sides to the helical support 34 of stainless steel shown here, which foam material is coated with active charcoal or nano composite material, where appropriate, or to which the latter is added. The PU foam forms a pore system the walls of which are coated with active charcoal so that a large material exchange surface is made available.

Concretely speaking, in the shown embodiment the support 34 consists of a VA grid member having a thickness of two to three millimeters, the helical structure being formed by two grid surfaces between which semi-hard, open-cell PU foam including an active charcoal coating is introduced. The grid bars 36 arranged on the downwardly directed side of the helix are provided with a photocatalytic surface, the mesh size being approx. 10 to 12 mm at these downwardly directed large surfaces. At the grid bars forming the upwardly directed large surface of the helix no coating is provided. The mesh size in this case is approx. 25 to 30 mm.

The micro-organisms and enzymes mentioned in the beginning can be introduced to the center of the helical packing 24 via a dosing hose. However, it is also possible to introduce these micro-organisms and enzymes including nano composite materials into the pore system already when manufacturing the packing 24. Tests were very promising in which the micro-organisms or enzymes and nano composite materials are dissolved in chitosane and this mixture to which the nano composite materials are added is then applied to the packing—for instance by soaking—so that continuous supply of micro-organisms or enzymes is superfluous and merely at regular intervals an exchange of the packing 24 is required.

The PU foam is coated with a gel-like material of chitosane on the downwardly directed side of the helix in the embodiment shown here. The nano composite materials are embedded in said chitosane which shows a respective piezoelectric ceramics system of PZT short fibers having photocatalytic coatings. Furthermore, micro-organisms responsible for fermentation or Kluyveromyces yeasts producing lactase are also embedded.

The container 22 is coated with the photocatalytically active layer 26—namely titanium dioxide, for instance—both at its inner surface and at its outer surface. This layer is completely applied to the inner surface, i.e. to the side facing the packing 34, while to the outer surface the titanium dioxide is applied in the form of strips 26 between which areas that are provided with a diamond coating 38 are retained.

Such diamond coating 38 can be synthetically prepared by heating methane and hydrogen as well as an appropriate carrier substance of niobium, silicon or ceramic, for example, in a vacuum chamber to temperatures of up to approximately 2000° C. Then a reaction takes place in which a diamond lattice is formed on the carrier substance. This coating 38 is then applied to the outer wall 28 of the container 22 so that areas provided with a photocatalytically active layer 26 and with a diamond layer 38 are juxtaposed. These areas 26, 38 extend in the longitudinal direction of the container 22. In the shown embodiment the width of the strips 26 corresponds approximately to the distance of four hole-shaped breakthroughs 30, while the width of the areas 38 is substantially smaller and corresponds approximately to the distance between two adjacent breakthroughs 30.

During interaction with the catalytic coating of the container 22 and the afore-described coating and the active charcoal of the helical packing 24 provided therein a comparatively strong electromagnetic field is formed. The occurring potential difference is applied to the areas provided with the diamond coating 38 which then act as diamond electrodes. This voltage can be used for separating the copper ions contained in the whey at the electrode acting as anode. In addition, also an external power source can be provided which actively supports the electrolysis of copper. Details about the electromagnetic field formed by the coating are disclosed in the earlier application DE 103 30 959.4 so that respective further explanations can be dispensed with.

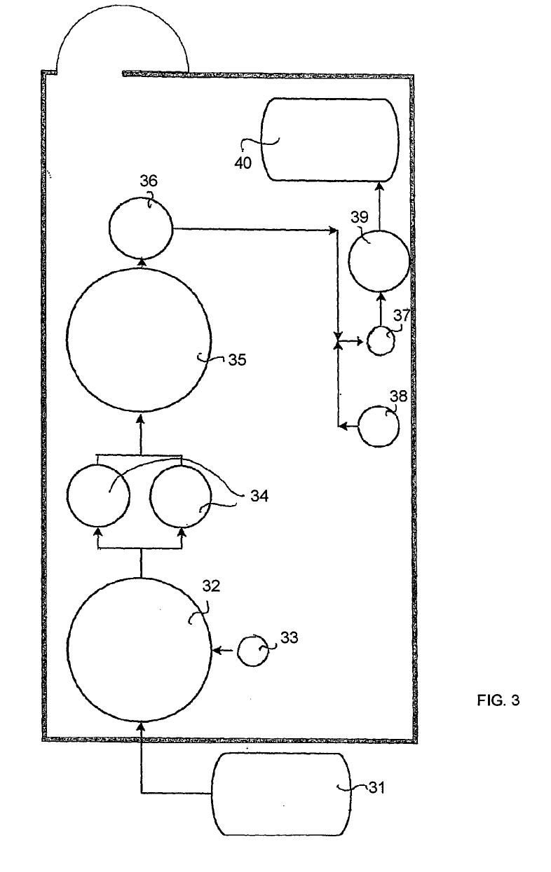

FIG. 3 schematically shows a further embodiment of the present invention in which ethanol is obtained from whey. From a tank 31 containing raw whey to which hop is added at a ratio of preferably 100 g/hl the whey is introduced into an aerobic loop reactor 32 of the type already described in the foregoing. If the whey is strongly loaded, the microbiotic mixture 33 described in the beginning can be added to the whey in the aerobic loop reactor.

Moreover, in the loop reactor a demineralization of the whey can take place. Especially potassium, but also salts, such as NaCl, for instance, influence the fermentation and can partly strongly impede the same.

The whey treated in this manner is then transferred into a buffer tank 34 in which the whey from the loop reactor is collected so as to be available for the further process. Whey can be combined from the most various dairy factories so that advantageously furthermore the whey is stabilized. It is especially advantageous in this context when whey having particular lactose content is provided in the buffer tank. To this effect, the introduced whey can be either diluted or concentrated.

In a further container 35 the pH value of the whey is raised to the value required for the subsequent enzyme treatment of between pH 5 to 7.5, especially pH=5.8 to 6.3. The enzyme treatment and the splitting of lactose and galactose into glucose have already been described in the foregoing.

Hereinafter in a membrane filtration plant 36 further possibly provided micro particles are removed from the whey so as to obtain a particularly good fermenting result.

The whey pre-treated in this manner—actually the fluid containing glucose in the meantime—is now transferred into a fermenting tank 37 in which fermenting yeast, which has preferably been pre-treated in a yeast fermenter 39, is added. After fermentation in a further tank 39 the yeast is deposited so as to obtain ethanol in an as pure form as possible. Then the ethanol obtained in this way is transferred into a further tank 40 in which the last residues of yeast and sugar are fermented into ethanol.

There is disclosed a method of decomposing fermentation-inhibiting substances in a fluid including saccharide-containing substances, wherein a microbiotic mixture, especially a mixture of photosynthetically working micro-organisms and light-emitting micro-organisms is introduced into the fluid, the mixture being intended to decompose the fermentation-inhibiting substances.

Although the best mode contemplated by the inventors of carrying out the present invention is disclosed above, practice of the above invention is not limited thereto. It will be manifest that various additions, modifications and rearrangements of the features of the present invention may be made without deviating from the spirit and the scope of the underlying inventive concept.

1. A method of decomposing fermentation-inhibiting substances in a fluid including saccharide-containing substances comprising introducing a microbiotic mixture of photosynthetically working micro-organisms and light-emitting micro-organisms into the fluid, the mixture decomposing the fermentation-inhibiting substances.

2. A method according to claim 1, wherein the microbiotic mixture is added to the fluid.

3. A method according to claim 1, wherein the saccharide-containing substances included in the fluid are pre-treated with the microbiotic mixture so as to be introduced into the fluid.

4. A method according to claim 1, wherein the fluid consists of food and/or food residues.

5. A method according to claim 4, wherein the fluid is whey.

6. A method according to claim 4, wherein the fluid is must or mush.

7. A method according to claim 6, wherein the substances forming the basis of the must and/or the barley are pre-treated with the microbiotic mixture.

8. A method according to claim 1, wherein the photosynthetically working micro-organisms contained in the microbiotic mixture are prochlorophytes, cyano bacteria, green sulphur bacteria, purple bacteria, chloroflexus-similar forms, helio bacteria and heliobacillus-similar forms as well as mixtures of two or more of them.

9. A method according to claim 1, wherein the light-emitting micro-organisms contained in the microbiotic mixture are photobacterium phosphoreum, vibrio fischeri, vibrio harveyi, pseudomonas lucifera or beneckea or mixtures of two or more of them.

10. A method according to claim 1, wherein the microbiotic mixture moreover contains plant extracts, enzymes, nutritional trace elements, polysaccharides, alginic derivatives and/or other micro-organisms either individually or in combination.

11. A method according claim 1, wherein hop and/or hop extracts are added to the fluid.

12. A method according to claim 1, wherein the method is used in ethanol preparation for decomposing fermentation-inhibiting substances.

13. A method of preparing ethanol, characterized in that a method according to claim 1 is carried out for decomposing fermentation-inhibiting substances.

14. A method of preparing ethanol from whey, characterized by the following steps of,
A. removing inhibitors from the whey by adding a microbiotic mixture of photosynthetically working micro-organisms and light-emitting micro-organisms;
B. transforming lactose contained in the whey into glucose; and
C. adding micro-organisms, especially yeast and/or bacteria for the preparation of ethanol from glucose.

15. A method according to claim 14, wherein the step of transforming lactose into glucose comprises the fact that lactose is split up into glucose and galactose and galactose is transformed into glucose by means of Kluyveromyces yeasts or organisms having a similar effect.

16. A method according to claim 14, wherein the inhibitors moreover contain copper which is removed from the whey by means of electrolysis.

17. A method according to claim 14, wherein prior to step B hop and/or hop extracts are added to the whey.

18. A method according to claim 14, wherein prior to step C a method of decomposing fermentation-inhibiting substances according to claim 1 is carried out.

19. A method according to claim 14, wherein in step C the content of ethanol is kept below 12%, by way of diffusion through an ethanol-permeable membrane.

20. A method according to any one of the claims 14, wherein step A is carried out at a temperature of between 20° C. and 70° C.

21. A method according to claim 14, wherein step B is carried out at a temperature of between 10° C. and 60° C.

22. A method according to claim 14, wherein step C is carried out, when using bacteria, at a temperature of between 30° C. and 80° C.

23. A method according to claim 14, wherein step C is carried out, when using yeasts, at a temperature of between 0° C. and 40° C., preferably at a temperature below 25° C.

24. A method according to claim 14, wherein the steps A, B and/or C are carried out successively in at least one bioreactor.

25. A method according to claim 24, wherein the at least one bioreactor (2a-c) comprises a container having at least one recess for the passage of the whey and a packing disposed in the interior of the container, wherein the outer walls of the container are coated in sections with a photocatalytically active layer and the packing has a coating of active charcoal.

26. A method according to claim 25, wherein a diamond coating is applied in the bioreactor used between the photocatalytically active coating provided in sections.

27. A method according to claim 25, wherein the photocatalytically active layer of the bioreactor used is titanium dioxide or indium tin oxide.

28. A method according to claim 25, wherein the photocatalytically active layer and the diamond layer are applied in the form of strips in the longitudinal direction of the container.

29. An arrangement of bioreactors (2a-c) for carrying out a method according to claim 14, wherein at least two, preferably three bioreactors are disposed in series and at least one of the bioreactors comprises a container having at least one recess for the passage of the whey and a packing disposed in the interior of the container, wherein the outer walls of the container are coated in sections with a photocatalytically active layer and the packing has a coating of active charcoal.

30. An arrangement of bioreactors according to claim 29, wherein a diamond coating is applied in the at least one bioreactor (2a-c) between the photocatalytically active coating provided in sections.

31. An arrangement of bioreactors according to claim 29, wherein the photocatalytically active layer of the bioreactor used is titanium dioxide or indium tin oxide.

32. An arrangement of bioreactors according to claim 29, wherein the photocatalytically active layer and the diamond layer are applied in the form of strips in the longitudinal direction of the container.

33. Use of a bioreactor for carrying out the method characterized in the claim 14 comprising a container having at least one recess for the passage of the whey and a packing disposed in the interior of the container, wherein the outer walls of the container are coated in sections with a photocatalytically active layer and the packing has a coating of active charcoal.

34. A method according to claim 24, wherein the at least one bioreactor is three bioreactors (2a-c) connected in series.
